(19) United States
(12) Patent Application Publication
(10) Pub. No.: US 2005/0149059 A1
(43) Pub. Date: Jul. 7, 2005

(54) Retinal flattener

(76) Inventor: Shibo Tang, Guangzhou (CN)

Correspondence Address: CARSTENS YEE & CAHOON, LLP, P O BOX 802334, DALLAS, TX, 75380, US

(21) Appl. No.: 11/008,010

(22) Filed: Dec. 9, 2004

(30) Foreign Application Priority Data
Dec. 19, 2003 (CN) 2003101174500

(51) Int. Cl. A61F 9/00

(52) U.S. Cl. 606/107

(57) ABSTRACT

The present invention relates to a retinal flattener, which is a medical device for retina reattachment in modern vitreoretinal surgery. The retinal flattener designed in the present invention generally contains a slender rod and a working tip at the forehead of the rod. The working tip consists of a base and a soft pressing element on the base. The special structures of the retinal flattener in the invention are designed according to the special requirement of the intraocular surgery. The form and the structure of the working tip facilitate operators to insert it into eyes from any of the two superior incisions in the modern standard three incisions vitreoretinal surgery. The soft pressing element applied in the working tip is used for retina-flatting operation such as rubdown and touch.

### TECHNICAL FIELD

The present invention relates to a medical device for retina reattachment in modern vitreoretinal surgery.

### BACKGROUND OF THE INVENTION

In modern vitreoretinal surgery, complete retinal reattachment is a sticking point to the success of the surgery. Along with the increasing maturation in microtechnique and the gradual growth in the understanding of the surgery, the problem how the retina, especially the macula and some other vital areas, could be better flatted and recovered after retinal detachment has a direct effect on the result of the whole surgery.

There has not been such a special device available used for that operation till now. Usually most operators use available retinal picks or flute needles to draw repeatedly on the retinal surfaces for the above purpose. This operation is inefficient and not very effective, and even worse, it is prone to retina hurt. Because the retina reattachment only depends on the repeated pull and draw, the occasions on which many operation traumas may be caused increase consequentially. Moreover, because of the metal devices, the retinal picks or the flute needles whose working surface consists of hard one can hardly avoid any small traumas to retinal neuroepithelial tissues and even would result in their complete tear.

### SUMMARY OF THE INVENTION

The purpose of the present invention is to design a special medical device, namely, a retinal flattener used for retina reattachment in the vitreoretinal surgery.

The retinal flattener designed in the present invention generally contains a slender rod and a working tip at the forehead of the rod, and a handle is implemented at the rear end of the rod for operators holding. The diameter of the rod ranges from 0.2˜0.9 mm. A certain angle which ranges form 120°˜150° and can be readjusted forms between the working tip and the rod. The working tip consists of a base and a soft pressing working element on the base. The base of the working tip is the extended part of the rod. The length of the whole working tip ranges from 0.5˜3.0 mm and its external diameter or width is not above 0.9 mm.

The special structures of the retinal flattener are designed according to the special requirement of the intraocular surgery. The form and the structure of the working tip facilitate operators to insert it into eyes from any of the two superior incisions in the standard three incisions vitreoretinal surgery. The soft pressing element applied in the working tip is used for retina-flatting operation such as rubdown and touch.

Generally said rod of the retinal flattener is a metal rod which is made of metal materials such as stainless steel or titanic alloy, and it also may be made of materials which have sufficient mechanical strength such as hard plastic and so on. The length of the rod which usually satisfies operators with length of 30˜40 mm is determined by the requirement of the surgery. Moreover, the soft pressing element on the working tip may use easy-shapable, flexible and elastic materials like silicone, rubber or artificial rubber, in which silicone is comparatively perfect.

The key part of the retinal flattener is the working tip, whose general structures in application are described below in two ways.

1. Said soft pressing element of the working tip is a piece of soft silicone film, which is inlaid in the side of the base and 0.05˜0.2 mm thick, and the portion of which extended from the base dimensions 0.05˜0.4 mm.

2. Said soft pressing element of the working tip is a silicone tube covered over the base, with its wall of 0.05˜0.2 mm thickness.

When the silicone tube is used in the soft pressing element, it should be a smooth joint between the silicone tube covered over the base and the abutting rod. The base of the working tip may consist of the core of a contracted rod extended from the front end of the rod, and the total external diameter of the working tip equals to the one of the rod while the silicone tube covers the base.

The retinal flattener designed in the present invention solves the problems like that traumas are often caused on the retina due to lack of suitable special device during the retina reattachment in modern vitreoretinal surgery, and that the effect of the surgery may be influenced and so on. Applying the retinal flattener of the present invention in vitreoretinal surgery we can benefit from following advantages:

1. At the later stage of tamponment of silicone oil or gas in the vitreoretinal surgery, the retina, especially the macula and some other vital areas, could be better flatted and recovered to improve the effect of the whole surgery;

2. During the relaxation and flatting of the retina, since applying the retinal flattener in the present invention with soft pressing element on the working tip in touching and rubbing down the retina, the operation times on the retina decreases and then the risk of traumas caused on the retinal surface due to devices themselves and repeated draw reduces greatly;

3. The duration of surgery can be saved and the operation efficiency can be raised.

### BRIEF DESCRIPTION OF THE DRAWINGS

FIG. 1 is a schematic structural illustration of the retinal flattener according to one of the embodiments in the present invention;

FIG. 2 illustrates an amplified view of the working tip in FIG. 1;

FIG. 3 illustrates the underside view of the working tip in FIG. 1;

FIG. 4 illustrates a section view in the direction A-A in FIG. 2;

FIG. 5 is a schematic structural illustration of the retinal flattener according to a second embodiment in the present invention;

FIG. 6 illustrates an amplified view of the working tip in FIG. 5;

FIG. 7 is a schematic structural illustration of the soft pressing element in FIG. 6 which is a silicone tube;

FIG. 8 is a schematic structural illustration of the base of the working tip in FIG. 6;

FIG. 9 is a schematic structural illustration of the retinal flattener according to a third embodiment in the present invention;

FIG. 10 illustrates an amplified view of the working tip in FIG. 9;

FIG. 11 is a schematic structural illustration of the soft pressing element in FIG. 10 which is a silicone tube;

FIG. 12 is a schematic structural illustration of the base of the working tip in FIG. 10.

### DETAILED DESCRIPTION OF THE PREFERRED EMBODIMENTS

Mode of Carrying Out the Invention

### EXAMPLE 1

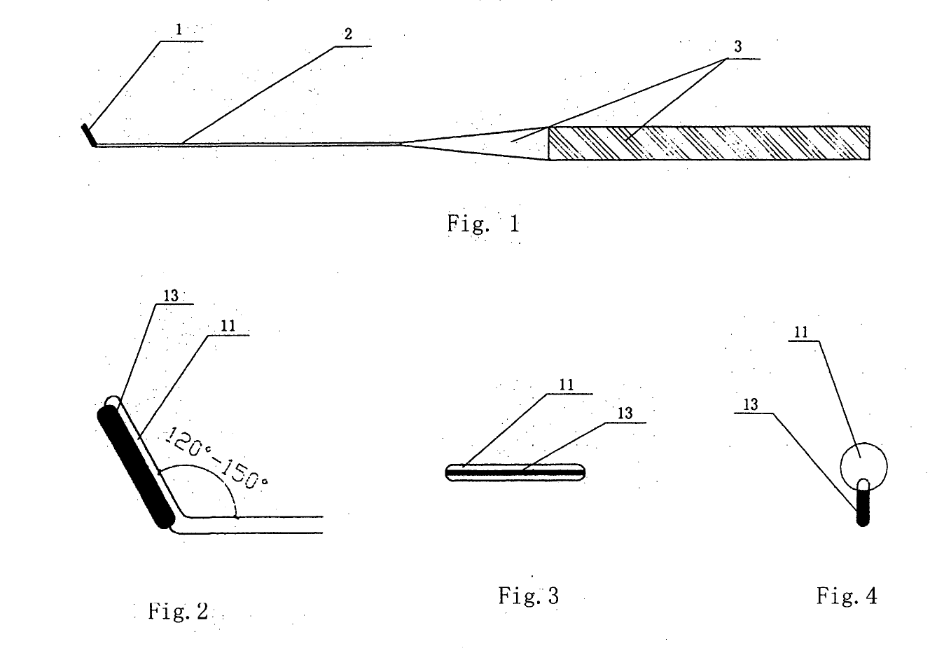

As shown in FIG. 1, the retinal flattener contains a working tip 1, a rod 2 and a handle 3, wherein the rod 2 is a metal rod made of stainless steel materials, whose diameter is 0.4 mm and length is 40 mm, and the angle between the working tip 1 and the rod 2 is 120°. The angle can be adjusted finely with hemostatic forceps in operation. The base 11 of the working tip, which is 2 mm long, is the extended part of the rod 2 with the same core (as shown in FIG. 2). A thin piece of silicone film 13, shaped like a brush, is inlaid underside the base 11 as a soft pressing working element. The soft silicone film 13 is 0.1 mm thick and the portion extended from the base dimensions 0.3 mm (as shown in FIGS. 3 and 4). The total length of the retinal flattener is about 110 mm.

When applying the retinal flattener in operation, at first an operator adjusts the angle of the working tip with hemostatic forceps according to the different areas rubbed down, and then, in order to flat the retina, inserts the device into an eye from any of the two superior incisions in the standard three incisions vitreoretinal surgery and tenderly rubs down the retinal surface responding to the area with the soft silicone film which is protruding from the working tip.

### EXAMPLE 2

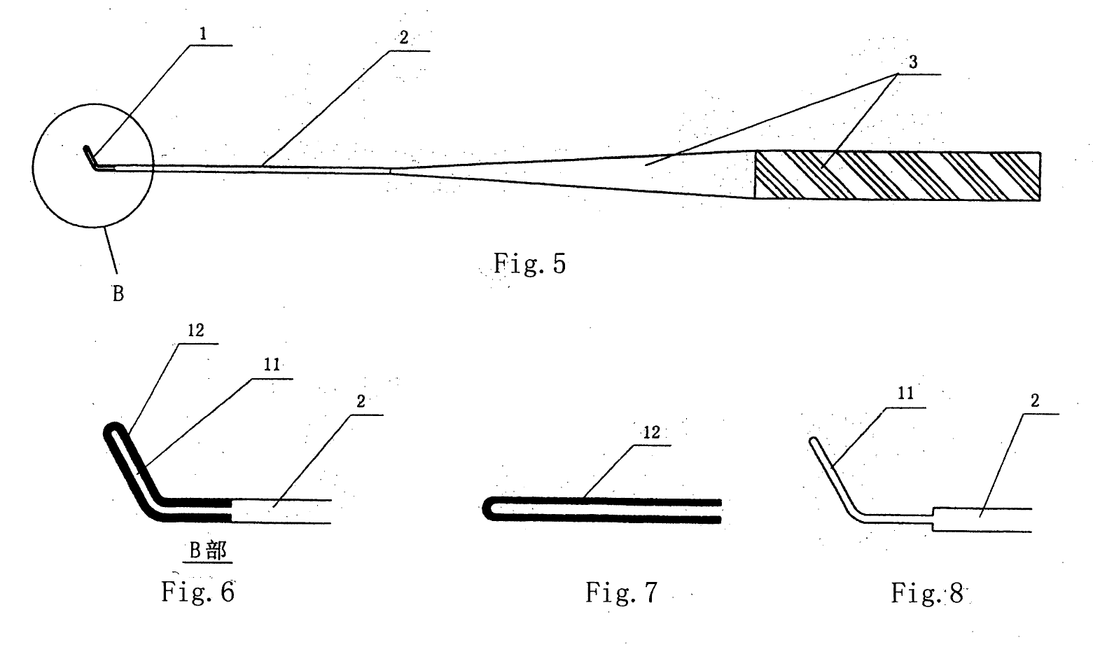

As shown in FIG. 5, the retinal flattener contains a working tip 1, a rod 2 and a handle 3, wherein the rod 2 is a metal rod made of stainless steel materials, whose diameter is 0.8 mm and length is 30 mm, and the angle between the working tip 1 and the rod 2 is 120°. The angle can be adjusted finely with hemostatic forceps in operation. The soft pressing element of the working tip is a silicone tube 12 covered over the base, whose wall is 0.2 mm thick (as shown in FIG. 7). The base of the working tip, whose diameter is about 0.4 mm, can be the core 11 of the contracted rod extended from the front end of the rod (as shown in FIG. 8). The total external diameter of the working tip equals to the one of the rod while the silicone tube covers the base (as shown in FIG. 6). The whole retinal flattener is about 130 mm long.

### EXAMPLE 3

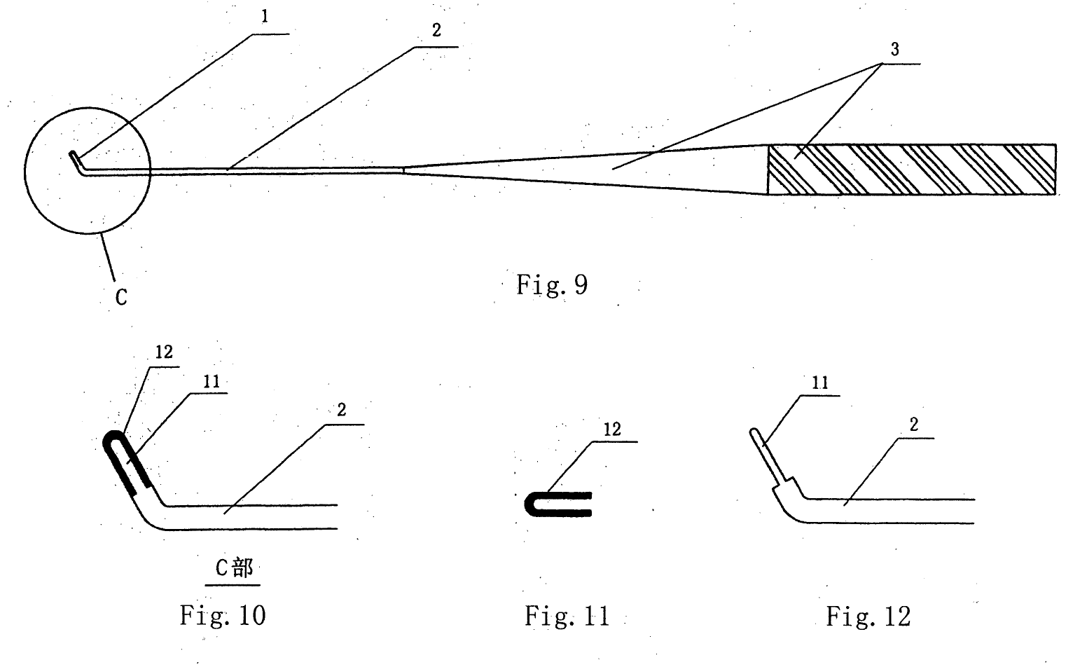

As shown in FIG. 9, the retinal flattener contains a working tip 1, a rod 2 and a handle 3, and its structure is the same as the one in Example 2 on the whole. The differences is that as the soft pressing element of the working tip, the silicone tube 12 only covers the front part of the working tip (as shown in FIG. 10). The silicone tube 12 and the base 11 of the working tip are shown in FIGS. 11 and 12.

1. A retinal flattener, characterized by a slender rod (2) whose diameter ranges from 0.2˜0.9, and a working tip (1) at the forehead of the rod, making a certain angle with the rod (2); the working tip (1) consists of a base (11) and a soft pressing element on the base; the base (11) of the working tip is the directly extended part of the rod (2); The length of the whole working tip ranges from 0.5˜3.0 mm and its maximal external diameter or width is not above 0.9 mm; and a handle (3) is implemented at the rear end of the rod (2).

2. A retinal flattener according to claim 1, wherein the soft pressing element of the working tip (1) is a piece of soft silicone film, which is inlaid in the side of the base (11) and 0.05˜0.2 mm thick, and the portion of which extended from the base dimensions 0.05˜0.4 mm.

3. A retinal flattener according to claim 1, wherein the soft pressing element of the working tip (1) is a silicone tube (12) covered over the base (11), with its wall of 0.05˜0.2 mm thickness.

4. A retinal flattener according to claim 3, wherein the base (11) of the working tip (1) is the core of a contracted rod extended from the front end of the rod (2), and the total external diameter of the working tip equals to the one of the rod (2) while the silicone tube covers the base (11).

5. A retinal flattener according to claim 3, wherein the angle between the working tip (1) and the rod (2) ranges from 120°˜150°.

6. A retinal flattener according to claim 3, wherein the rod (2) is a metal rod.

7. A retinal flattener according to claim 3, wherein the rod (2) is 30˜40 mm long.
